(19) United States
(12) Patent Application Publication
(10) Pub. No.: US 2008/0027519 A1
(43) Pub. Date: Jan. 31, 2008

(54) Method of treatment of ocular compartment syndromes

(76) Inventor: John M. Guerrero, Palm City, FL (US)

Correspondence Address: DARBY & DARBY (formerly Sacco & Associates), P.O. BOX 770, CHURCH STREET STATION, NEW YORK, NY, 10008-0770, US

(21) Appl. No.: 11/494,949

(22) Filed: Jul. 28, 2006

(51) Int. Cl. A61N 5/06

(52) U.S. Cl. 607/89

(57) ABSTRACT

A method of treatment of ocular compartment syndromes is provided. Known ocular compartment syndromes include central retinal vein occlusion (CRVO), branch retinal vein occlusion (BRVO), non-arteritic anterior ischemic optic neuropathy (NAAION), and papilledema. One or more lasers known to have photoablative, photodisruptive, or photocoagulative effects are used to decompress the ocular compartment syndromes. The method includes the steps of positioning a patient beneath an operative microscope (110) and one or more lasers (108,109,111), positioning a fixation ring (104) on the operative eye (102) identifying the site of the occlusion using an operative microscope (110), and directing laser energy at the target tissue responsible for the occlusion. A display (112) is provided to guide the surgeon performing the laser treatments. A microscope and laser control system (114) is provided to allow the surgeon to control the operative microscope (110) and the lasers (108,109,111).

### BACKGROUND OF THE INVENTION

1. Statement of the Technical Field

The invention relates generally to the treatment of ocular compartment syndromes, and more specifically, to a method and related operative arrangements using one or more lasers each having one of a photoablative, photocoagulative, or photodisruptive effect on target tissue for the treatment of the ocular compartment syndromes.

2. Description of the Related Art

A compartment syndrome is defined as the presence of increased pressure in a closed (usually fascial) space. As pressure within the enclosed space exceeds venous pressure, venous stasis may occur. When the pressure within the enclosed space exceeds arterial pressure or when compressive forces cause physical collapse of a vessel, arterial flow ceases and anoxia ensues. Although the term “compartment syndrome” is most often used in the setting of orthopedics, the fundamental physiology of compartment syndromes applies to several pathologies of the eye, including central retinal vein occlusion (CRVO), branch retinal vein occlusion (BRVO), non-arteritic anterior ischemic optic neuropathy (NAAION), and papilledema (swelling of the optic disc).

Central Retinal Vein Occlusion (CRVO)

The venous circulation of the retina drains to the ophthalmic veins in the orbit via the central retinal vein. The central retinal vein exits the eye by passing through the sclera along with the optic nerve. Central retinal vein occlusion is a condition in which blood flow through the central retinal vein is obstructed. The obstruction can be caused by a thrombus, or blood clot. Most of these occlusions occur as the central retinal vein passes through a structure known as the lamina cribrosa. It has been hypothesized that occlusions occur at this location because the lamina cribrosa is the site of greatest physical constriction and compression of the central retinal vein as it leaves the globe (eyeball) and enters the orbit (socket).

With age, blood vessel walls may thicken and become less compliant. In the area of the lamina cribrosa where there is little room for outward expansion, a vessel can become sufficiently compressed to interrupt blood flow. Even if the compression is insufficient to completely occlude the vessel, a focal narrowing of the vein results in local turbulent blood flow. Such turbulent flow is felt to contribute to thrombus formation which subsequently occludes the vein completely.

The lack of venous outflow from the retina causes stasis of retinal blood flow. This results in retinal edema (swelling) and poor visual function. Most patients who experience CRVO will have 20/400 or worse vision in the affected eye. Further complications are not uncommon as the lack of retinal blood flow can cause the release of chemical messengers known as angiogenic factors. These chemical messengers encourage the growth of new blood vessels (neovascularization). Although in theory this sounds desirable, neovascularization does not restore normal retinal blood flow. The fragile and inappropriately located new vessels often hemorrhage, resulting in scarring, retinal detachment, and further loss of vision. When neovascularization develops in the trabecular meshwork (the site which controls the intraocular pressure or inflation pressure of the eye), a rapid increase in intraocular pressure (IOP) often results. This condition is known as neovascular glaucoma and can result in total loss of vision as well as severe pain which may require removal of the diseased eye.

CRVO is usually a diagnosis followed by an apology, as no reliable vision-improving treatment is available. Management is directed towards preventing neovascular complications by frequent surveillance and pan-retinal photocoagulation (PRP) to abort neovascularization should it occur.

Physicians have used various techniques in an attempt to restore venous drainage and hopefully improve vision or at least reduce the risk of neovascularization. Chorioretinal anastamosis was one such technique. The goal of chorioretinal anastamosis is to create a vascular shunt between the retinal venous circulation and the underlying choroidal circulation. This is accomplished through the application of laser energy (usually in the 400 nanometer to 800 nanometer spectrum) to puncture a hole through a retinal vein, through the underlying retina and through the retinal pigment epithelium into the choroid. This technique was fraught with complications and even when anatomically successful generated little or no clinical benefit.

Recently, emphasis has been placed on reopening the occlusion in the central retinal vein rather than by trying to create an artificial bypass around it. In one such technique, instruments are passed through small incisions made in the anterior eye wall. These instruments are first used to perform a vitrectomy or surgical removal of the vitreous from the eye. The vitreous is a viscous, tenacious, gel-like substance that fills the posterior chamber of the eye and adheres to the surface of the retina. If instruments are maneuvered in the posterior chamber without first removing the vitreous, the instruments can engage the vitreous and pull on the retina which may result in retinal tears, retinal edema, and retinal detachment.

Following vitrectomy, a tiny catheter is used to canulate the central retinal vein and inject a clot-lysing agent. During the same procedure, the catheter may be advanced through the lumen of the vessel in an attempt to mechanically disrupt the clot and dilate the vessel lumen. This technique has enjoyed only limited success and carries all the risks of intraocular surgery including, but not limited to, infection, hemorrhage, and retinal detachment. Furthermore, the procedure is very challenging to perform and avulsions or lacerations of the retinal vasculature as well as collateral damage to surrounding structures are not uncommon. This technique also fails to address the actual “compartment” of the compartment syndrome. The anatomical narrowing of the central retinal vein as it passes through the lamina cribrosa still remains, thereby leaving a nidus for future clot formation and recurrent venous occlusion.

Another technique, known as radial neurotomy, does address the issue of focal narrowing of the central retinal vein as it passes through the lamina cribrosa. In this approach, a vitrectomy is performed to allow instruments to be manipulated in the posterior segment of the eye. An incising device (such as a steel or diamond blade on an appropriate handle) is used to create a radial incision in the optic nerve deep enough to incise the lamina cribrosa in the area through which the central retinal vein courses. This serves to decompress the central retinal vein and thereby restore venous outflow. This procedure carries all of the risks of intraocular surgery and is difficult to perform. The area being perforated is exquisitely delicate as are the surrounding structures which include the central retinal vein itself, the central retinal artery, and the nerve fibers of the optic nerve. Collateral damage to these structures is not uncommon.

A preferred solution to this compartment syndrome would be a technique that would allow a more controlled decompression of the central retinal vein with less risk of damage to the surrounding structures. Ideally this technique would not require traditional incisional intraocular surgery.

Branch Retinal Vein Occlusion (BRVO)

Branch retinal vein occlusions (BRVO's) represent a blockage in retinal venous flow prior to the level of the central retinal vein. Like central retinal vein occlusions, branch occlusions result in retinal hemorrhage, edema, and vision loss. Visual loss from a BRVO is often less severe than the visual loss caused by a CRVO. Likewise, neovascular complications are less frequent with a branch occlusion than with a central retinal vein occlusion.

Most branch retinal vein occlusions occur where a retinal artery passes over (or under) a retinal vein. At these arteriovenous crossings, the artery and vein are surrounded by a connective tissue enclosure which allows for very little expansion of either vessel. With advancing age and atherosclerosis, the walls of the retinal arteries thicken and become less compliant. Trapped within a common facial sheath, the hardened retinal artery begins to compress the underlying vein and a compartment syndrome develops. The kink or nick produced in the vein can be so severe that it blocks all venous flow through the vessel. Alternatively, turbulent blood flow through a compressed and narrowed vein can promote clot formation with the resulting thrombus completing the venous occlusion within the fascial compartment.

One approach to the treatment of branch retinal vein occlusions involves the canulation of the affected vessel and injection of clot-lysing agents. Attempts have also been made to surgically decompress the affected vein by lysing the fascial sheath that binds the artery and vein together. The internal limiting membrane (ILM) of the retina is occasionally removed as well. All of these techniques suffer from similar drawbacks to those associated with the surgical decompression of central vein occlusions, namely the attendant risks of intraocular surgery, the inherent difficulty of the procedure, and the very real risks of damage to surrounding structures.

Accordingly, a technique which would allow more controlled decompression of a branch retinal vein with less risk of damage to the surrounding structures would be preferred. Ideally this technique would not require intraocular surgery so as to avoid the attendant risks associated therewith.

Non-Arteritic Anterior Ischemic Optic Neuropathy

Although not a retinal vascular occlusion in the traditional sense, Non-Arteritic Anterior Ischemic Optic Neuropathy (NAAION) seems to share the same compartment syndrome etiology as Central Retinal Vein Occlusion (CRVO) and Branch Retinal Vein Occlusion (BRVO). In this condition, there is an interruption of blood flow to the small vessels which supply the anterior portion of the optic nerve. Vision loss in NAAION is painless, rapid, and permanent. Risk factors for NAAION include atherosclerosis (as this impairs blood flow through the blood vessels which supply the optic nerve) and a “tight” optic nerve. Also called “a disc at risk”, an optic nerve with a small or absent optic cup makes a “tight” passage through the sclera as it enters the eye. This tight passage through the sclera is felt to place further pressure on the small vessels that supply the optic nerve. As atherosclerosis causes an increase in the outer diameter (and a decrease in the inside diameter) of these small vessels, there is no room for the vessels to expand as they are confined by the “tight” optic nerve. This process eventually leads to a loss of adequate blood flow to the optic nerve and Ischemic Optic Neuropathy ensues. This is analogous to the situation in CRVO in which the central retinal vein makes a tight passage through the lamina cribrosa. As with CRVO, attempts to treat NAAION have included radial neurotomy in order to relieve the mechanical pressure on the optic nerve and its supporting vasculature. Radial neurotomy for NAAION is fraught with the same risks and difficulties as radial neurotomy used in the treatment of CRVO (described above).

Accordingly, a preferred solution to the problem would be a technique which would allow more controlled decompression of the optic nerve with less risk of damage to the surrounding structures. Ideally this technique would not require traditional incisional intraocular surgery.

Surgical Lasers

Ophthalmic surgery currently makes use of a large array of surgical lasers to treat a variety of ocular diseases. Whereas physicists classify lasers according to the lasing medium and/or the physical properties of the emitted radiation, physicians more often classify lasers according to the effect they have on a target tissue. Ophthalmic lasers are generally considered to be photocoagulative, photodisruptive, or photoablative.

When a photoablative laser interacts with human tissue, the laser energy interacts with the target tissue at the molecular level. The laser energy causes molecular bonds in the target tissue to be blown apart. The result is the ablation of the targeted tissue. In ophthalmic surgery, the most commonly used photoablative laser is a nanoseconds duration excimer laser radiating in the UV spectrum (193 nm). In general, photoablative lasers can accomplish very precise and reproducible tissue removal. The excimer lasers routinely used in ophthalmic surgery are capable of reliably removing tissue in 0.25 micron increments.

It is important to clarify that other lasers can exhibit similar photoablative properties. The femtosecond infrared laser, for example, demonstrates excellent photoablative properties although it is often technically considered a photodisruptive laser. Laser photoablation already enjoys extensive ophthalmic use in refractive surgery as it allows controlled removal of tissue with exquisite precision, negligible thermal damage, and negligible disturbance of surrounding tissues.

Photodisruptive lasers enjoy routine use in ophthalmic surgery and are most commonly used to open a cloudy posterior capsule following cataract extraction/intraocular lens implantation surgery. In this instance, laser energy interacts with the fluid immediately in front of (or immediately behind) the target tissue. When the laser energy interacts with this fluid, a tiny cavitation bubble is created. As the cavitation bubble collapses, a minute shock wave is created which propagates through the fluid and creates the desired tear in the posterior capsule. This procedure is most commonly achieved with a nanosecond duration Neodymium Yttrium Aluminum Garnet (Nd:YAG) laser emitting in the infrared (1064 nanometer) spectrum. Photodisruptive lasers are also used to perform peripheral iridotomies for the prevention or treatment of angle closure glaucoma.

Although photodisruption with the traditional Nd:YAG laser offers less control over tissue removal than excimer laser photoablation, photodisruption with femtosecond lasers (such as a 1064 nm, infrared, fs pulse width device) offers exquisite control over tissue disruption. In this regard, the femtosecond infrared laser, although technically a photodisruptive laser, demonstrates properties much like the photoablative excimer laser. The femtosecond infrared laser's high degree of control is coupled with minimal damage to surrounding tissues due to the short duration of the laser applications.

When a photocoagulative laser interacts with a target tissue, pigments in the tissue (called chromophores) absorb the laser energy. The absorbed laser energy is converted to heat, which subsequently denatures (coagulates) the proteins in the target tissue. Photocoagulation of human tissue can be accomplished with a gas (usually argon) laser, a diode laser, or a frequency-doubled Nd:YAG laser, generally emitting in the 400-800 nanometer spectrum. Laser photocoagulation is routinely used in ophthalmology for the treatment of a variety of conditions including diabetic retinopathy, retinal tears, and glaucoma.

As outlined above, ophthalmic lasers are better described by their interactions with a target tissue rather than by their physical construction. An Nd:YAG laser, for example, can be photocoagulative when used with a frequency doubler (532 nm). An Nd:YAG laser can be photodisruptive when used at nanosecond durations and with a wavelength of 1064 nm. When the same 1064 nm Nd:YAG laser is used at femtosecond durations, it exhibits properties characteristic of photoablation.

It is important to note that in ophthalmic surgery, lasers with different properties may be combined in order to achieve a desired therapeutic effect. When performing a peripheral iridotomy (PI), for example, many surgeons, will use both a photocoagulative laser (such as a diode laser) and a photodisruptive laser (such as a nanosecond Nd:YAG laser emitting at a wavelength of 1064 nm) to perform the procedure. The photocoagulative laser is used to thin the iris stroma and coagulate local blood vessels. The photodisruptive laser is then used to punch through the thinned stroma and complete the iridotomy.

### SUMMARY OF THE INVENTION

The invention concerns a method and related operative arrangement for using laser energy to ablate, incise, disrupt, or otherwise relax a tissue of the body which is restricting ocular blood flow by constricting or compressing an ocular structure such as a blood vessel. Although these methods and devices are particularly suited to the treatment of retinal vein occlusions and non-arteritic anterior ischemic optic neuropathy in human eyes, they are not necessarily limited to these applications. For example, the methods and devices described may be utilized in animals, or for other vascular occlusions such as arterial occlusions. This treatment may also find use in decompression of the optic nerve and optic nerve sheath in cases of papilledema.

The method begins with the step of positioning a patient so that ocular tissue can be observed with an operative microscope. The patient's operative eye is then immobilized to prevent movement of the operative eye during the treatment. The method also includes the step of identifying the site of the occlusion responsible for the compartment syndrome. Thereafter, the method can include directing one or more different types of laser energy at an ocular tissue that is identified as the source, or is causing or is otherwise responsible for an ocular compartment syndrome. In particular, the laser energy can be directed at an ocular tissue to relieve at least one symptom of the ocular compartment syndrome. The patient can be positioned so that the ocular tissue is exposed to the laser energy. The laser energy can be selected based on the desired effect on the ocular tissue. For example, the laser energy can be selected for causing an effect that is (a) photoablative (b) photodisruptive and/or (c) photocoagulative.

If a photoablative effect is desired, a photoablative laser such as a femtosecond duration Nd:YAG laser, is selected. Such an Nd:YAG laser can radiate laser energy at about 1064 nanometer wavelength with pulse durations of femtoseconds to hundreds of femtoseconds.

Other types of lasers can also be used for tensioning and producing photocoagulation of the ocular tissue. For example, a gas (usually argon) or diode laser can be used for this purpose. The laser used for photocoagulation can produce laser energy with a wavelength in the range from 400 to 800 nanometers. Moreover, photodisruptive lasers can also be used to incise the target ocular tissue. For example, a nanosecond duration, 1064 nm Nd:YAG laser can be used for this purpose.

In the case of a central retinal vein occlusion (CRVO), laser energy is directed at the site of compression of the central retinal vein, generally at the level of the lamina cribrosa. In particular, the ocular tissue targeted for application of laser energy can include a portion of the optic nerve of the operative eye. For example, the laser energy can be used for incising the head of the optic nerve. A photocoagulative laser can be used to thin the target tissue, place the tissue under tension, and/or to control bleeding. In addition, a photodisruptive laser may be used to incise the lamina cribrosa. A photoablative or a photodisruptive laser with photoablative properties can be used to ablate the lamina cribrosa so as to decompress the compartment which is compressing the central retinal vein.

In the case of a branch retinal vein occlusion (BRVO), laser energy is directed at the area of the branch vein occlusion. Most commonly, this will be at an arterio-venous crossing. For example, a fascial sheath and/or an internal limiting membrane (ILM) surrounding an area of retinal venous constriction can be targeted for application of laser energy. Laser energy can be used to disrupt or ablate the fascial sheath that binds the artery to the vein. A photocoagulative laser can be used to thin the fascial sheath and/or ILM, or to place these tissues under tension so as to facilitate their disruption or ablation by another laser. The photocoagulative laser can be also used to control bleeding. In addition, a photodisruptive laser may be used to incise the fascial sheath and or ILM. A photoablative or a photodisruptive laser with photoablative properties can be used to ablate the fascial sheath in the area of the BRVO.

According to yet another aspect of the invention, the ocular tissue selected for application of laser energy can be a site of a non-arteritic anterior ischemic optic neuropathy (NAAION). In the case of NAAION, laser energy is directed at the optic nerve or optic nerve sheath. Particularly, the laser can target a thin radial strip of the substance of the optic nerve. The incision can be carried through the optic nerve head, preferably through the level of the lamina cribrosa. The nerve can generally be incised at the nasal midline in order to minimize visual field loss and avoid macular nerve fibers. A photocoagulative laser can be used to thin the target tissue and/or to place the tissue under tension. The photocoagulative laser can also be used to control any bleeding during the surgery. Incision of the optic nerve head can then be completed with a photoablative laser (or a photodisruptive laser with photoablative properties) and/or a traditional photodisruptive laser.

In the case of papilledema, laser energy is directed at the optic nerve rim. The goal is to decompress the optic nerve or the optic nerve sheath. A photocoagulative laser can be used to thin the nerve rim and place the target tissue under tension. The photocoagulative laser can also be used to control bleeding during surgery. A photoablative or a photodisruptive laser with photoablative properties can be used to incise the nerve rim.

### BRIEF DESCRIPTION OF THE DRAWINGS

FIG. 1 is a block diagram of an operative arrangement for a method of treatment of ocular compartment syndromes using a single laser source that is useful for understanding the invention.

FIG. 2 is a block diagram of an operative arrangement for a method of treatment of ocular compartment syndromes using a second laser source that is useful for understanding the invention.

FIG. 3 is a block diagram of an operative arrangement for a method of treatment of ocular compartment syndromes using a third laser source and a high-resolution tomographer that is useful for understanding the invention.

FIG. 4 is a flow diagram of a method of treatment of a central retinal vein occlusion that is useful for understanding the invention.

FIG. 5 is a flow diagram of a method of treatment of a branch retinal vein occlusion that is useful for understanding the invention.

FIG. 6 is a flow diagram of a method of treatment of non-arteritic anterior ischemic optic neuropathy (NAAION) that is useful for understanding the invention.

FIG. 7 is a flow diagram of a method of treatment of papilledema that is useful for understanding the invention.

### DETAILED DESCRIPTION OF THE PREFERRED EMBODIMENTS

The present invention makes use of laser photoablation, photodisruption, photocoagulation, or a combination thereof, in order to decompress an ocular compartment syndrome. In the case of a central retinal vein occlusion, laser energy is directed at the site of vascular compression, usually at the level of the lamina cribrosa. In the case of branch retinal vein occlusions, laser energy is delivered to the fascial sheath which binds the retinal vein to its companion artery so as to decompress the site of venous compression. In the case of non-arteritic anterior ischemic optic neuropathy (NAAION) or papilledema, the laser energy is directed at the optic nerve and/or nerve sheath in much the same way a surgical blade would be directed at the nerve in order to perform a traditional surgical radial neurotomy.

The advantageous qualities of photoablation make the process desirable for the controlled decompression of the lamina cribrosa in the area of an occluded central retinal vein. Likewise, for branch retinal vein occlusions in which a retinal vein is compressed by an adjoining artery as they pass through their common fascial sheath, laser photoablation can be used to ablate the fascial layer thereby releasing the compressive forces on the involved retinal vein.

Published research has suggested that surgical delamination of the internal limiting membrane (ILM) in the area of a BRVO at the same time as decompression of the fascial sheath may improve final visual outcome. To this end, laser photoablation could also be used to locally ablate the ILM in the area of a BRVO without the need for intraocular surgery. For the treatment of NAAION or papilledema, photoablation can be used to create a precise incision in the optic nerve with far greater control and far less risk to adjacent structures than a radial neurotomy performed with a hand-held surgical knife blade. By creating the smallest possible incision required to produce a therapeutic effect, radial neurotomy performed with a laser will cause less loss of nerve fibers than radial neurotomy performed with a blade.

Tissue ablation with a photodisruptive laser such as a femtosecond infrared device is well suited for the controlled decompression of the lamina cribrosa in the area of an occluded central retinal vein. Likewise, for branch retinal vein occlusions, laser photodisruption can be used to ablate the fascial sheath and/or Internal Limiting Membrane that is compressing a retinal vein. Published research has suggested that surgical delamination of the internal limiting membrane (ILM) in the area of a BRVO at the same time as decompression of the fascial sheath may improve final visual outcome. To this end, laser photodisruption could be used to locally ablate the ILM in the area of a BRVO without the need for intraocular surgery.

For the treatment of NAAION or papilledema, photodisruption can be used to create a precise incision in the optic nerve (neurotomy) with far greater control and far less risk to adjacent structures than a radial neurotomy performed with a handheld surgical knife blade. By creating the smallest possible incision required to produce a therapeutic effect, a neurotomy performed with a laser results in less loss of nerve fibers than radial neurotomy performed with a blade.

Although laser photocoagulation offers far less control over tissue removal than laser photoablation or photodisruption, it can be used to decompress a central or branch retinal venous occlusion either alone or in conjunction with a photoablative and/or photodisruptive laser. In this regard, laser photocoagulation would be most useful in arresting any bleeding caused by the use of a photoablative or photodisruptive laser in the treatment of ocular compartment syndromes. Since laser photocoagulation generally causes tissue shrinkage, a photocoagulative laser can also be used to place a tissue under tension prior to treatment with a photodisruptive and/or photoablative laser.

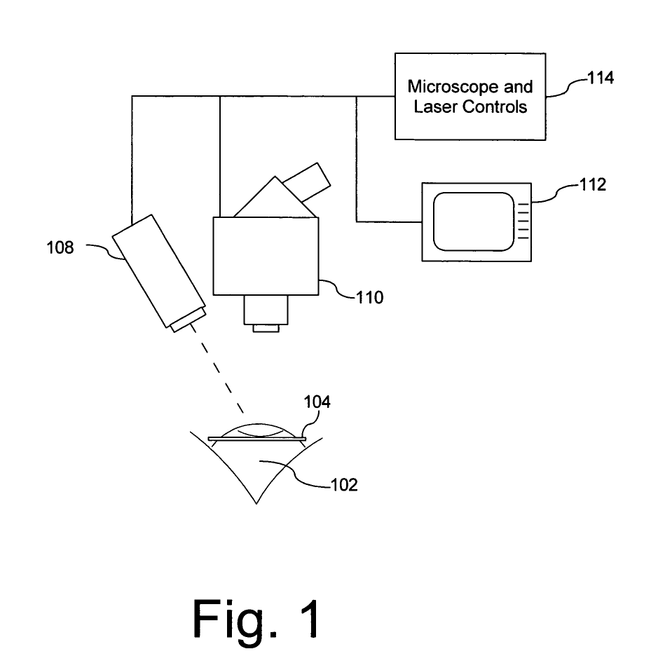

Referring now to FIG. 1, shown is an arrangement of surgical equipment that can be used for implementing a method of treating ocular compartment syndromes that is useful for understanding the invention. In the preferred embodiment of the invention, the arrangement includes an eye fixation ring 104 or similar device which mechanically steadies the patient's operative eye 102 in the focal path of an operating microscope 110 and a laser 108. The selection of the eye fixation ring 104 as the means for steadying the patient's eye is not limited in this regard as any one of several means well known in the art can be utilized. The operating microscope 110 and laser 108 are controlled by a microscope and laser control system 114. The image of the operative eye 102 formed on the lenses (not shown) of operating microscope 110 that is the subject of the laser treatments is displayed on display 112 to aid the surgeon performing the laser treatments as described more fully hereinbelow.

The laser 108 can be selected to include any suitable laser for causing a photoablative and/or a photodisruptive effect. In the preferred embodiment of the invention, laser 108 can be selected to be a photoablative laser including a Nd:YAG laser radiating in the infrared range (1064 nm) with a pulse duration measured in nanoseconds to tens of nanoseconds. In an alternate embodiment of the invention, the laser 108 can be selected to be a photoablative laser including a Nd:YAG laser radiating in the infrared range (1064 nm) with a pulse duration measured in femtoseconds to hundreds of femtoseconds. Those skilled in the art will appreciate that the Nd:YAG laser can be photodisruptive when used at a nanosecond durations with a wavelength of 1064 nm. When the same 1064 nm Nd:YAG laser is used at femtosecond durations, it can produce effects which are on the border between the effects produced by the photodisruptive and photoablative lasers. Still, it should be understood that the method disclosed herein is not limited to the particular types of lasers and/or pulse durations described herein. Instead, any type of laser can be used that is capable of causing a desired photoablative, photodisruptive and/or photocoagulative effect.

The energy level selected for use with the laser 108 used with the present invention will vary greatly based on the type of laser used and the tissue being targeted. Incising the optic nerve head, for example, would be expected to require laser energy on an order of magnitude higher than the laser energy required for incising the ILM (internal limiting membrane). The clarity of the patient's ocular media will also affect the laser energy needed to complete a procedure. For example, incising the optic nerve head in a patient with a dense cataract will take far more laser energy than incising the optic nerve head in a patient with a clear/non-cataractous lens. In general, a photocoagulative laser such as a green diode laser would be expected to utilize spot sizes between 25 and 500 microns. The duration of the laser treatments selected would vary between milliseconds bursts and a continuous wave. The power level selected for the laser treatments would be in the 50 milliwatt to 1 watt range. For a traditional nanosecond(s) duration photodisruptive Nd:YAG laser, energy delivery would vary between millijoules and hundreds of millijoules per pulse. For a photodisruptive femtosecond(s) duration Nd:YAG laser, energy fluence would vary between tens of joules per square centimeter and thousands of joules per square centimeter.

The operative site, including the patient's operative eye 102, can be visualized by the surgeon by selecting a safety-shielded optical or videographic electronic display 112. Still, the selection of the display for viewing the operative site is not limited in this regard. The control of the microscope (zoom, focus, X-Y, tilt, brightness, etc.) and laser (focal point, power, spot size, spot shape, spot pattern, etc.) are performed from microscope and laser controls 114 using established control techniques.

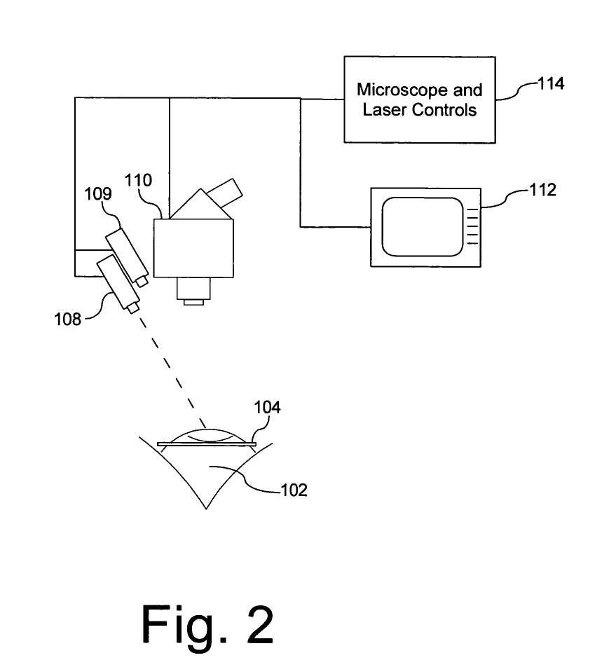

Referring now to FIG. 2, shown is an alternate embodiment of an arrangement of surgical equipment that can be used for implementing the method of treating an ocular compartment syndrome that is useful for understanding the invention. The common features shown in FIG. 2 are identified using the same reference numerals as previously used in to FIG. 1. Thus, FIG. 2 includes an eye fixation ring 104, operating microscope 110, laser 108, optical or electronic display 112 and microscope/laser controls 114 as previously discussed. In addition, a second laser source 109 has been added. By combining and selecting lasers which each have a different effect on target tissue (i.e. photoablative, photocoagulative, and photodisruptive) decompression of ocular compartment syndromes can be performed with greater safety and efficacy. In the preferred embodiment of the invention, the first laser source 108 can be selected to be the photoablative laser previously described. The second laser source 109 can be selected to be a photocoagulation diode laser producing laser energy with a wavelength in the range of 400 to 800 nanometers. The photocoagulation laser can be used for tensioning and/or thinning a target tissue by photocoagulation of the target ocular tissue. However, the invention is not limited to this specific range of wavelengths and any other laser energy can be selected provided that it can produce the desired tensioning or photocoagulation of ocular tissue.

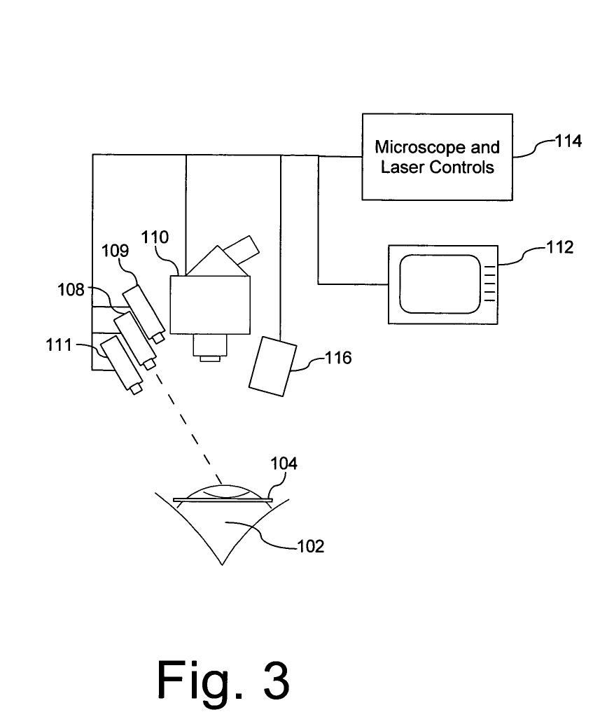

Referring now to FIG. 3, shown is another embodiment of an arrangement of surgical equipment that can be used for implementing a method of treating ocular compartment syndromes that is useful for understanding the invention. The arrangement in FIG. 3 adds a third laser source 111, so that all three of the previously described laser types can be selected including the photocoagulative, photodisruptive, and photoablative lasers for use in the laser treatments. This provides maximum versatility in the treatment of ocular compartment syndromes, including the management of intra-operative hemorrhage. In addition to the operating microscope 110, a high resolution imaging system 116, such as an Optical Coherence Tomographer (OCT) has also been added. The additional resolution provided by this high resolution imaging system 116 provides augmented microscopic visualization of the treatment area and gives the surgeon a clearer view of the effect that the laser is having on the target tissue. This allows better titration of therapy and less damage to tissues surrounding the treatment area.

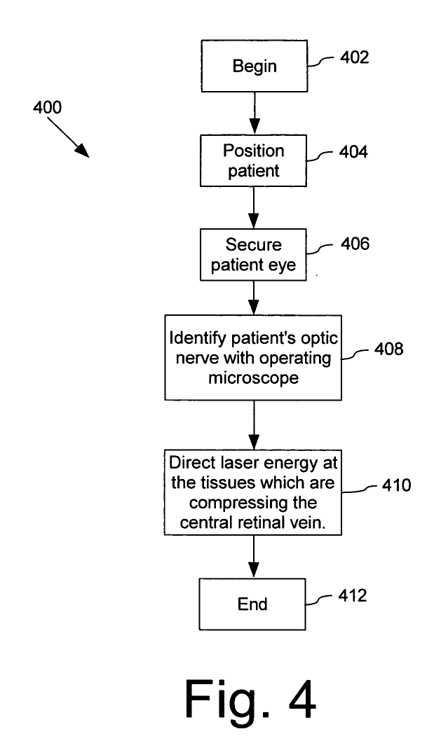

Referring now to FIG. 4, shown is a flow diagram of a method of treatment 400 for ocular compartment syndromes such as a central retinal vein occlusion (CRVO) that is useful for understanding the invention. As discussed earlier, CRVO generally occurs in the area where the central retinal vein enters the globe through the lamina cribrosa of the optic nerve. The vein makes a tight fit as it passes through a fenestration in this connective tissue structure. As the vessel wall thickens with age, it is trapped within this connective tissue compartment and becomes compressed, eventually compromising blood flow.

The method of treatment 400 begins with step 402 and continues with step 404. In step 404, the patient is positioned beneath an operative microscope 110 and one or more of lasers 108, 109, and 111. In step 406, the operative eye 102 is stabilized by selecting and positioning a fixation ring 104 or other fixation device on the operative eye 102. In step 408, microscopic visualization is used to identify the patient's central retinal vein as it passes through the optic nerve. In step 410, laser energy is directed at the tissues which are compressing the central retinal vein. This step involves selecting one or more of lasers 108, 109, and 111 depending upon the effect desired. A single photocoagulative, photodisruptive, or photoablative laser may be selected. However, in the preferred embodiment of the invention, a combination of one or more laser types is selected in order to achieve the desired effect. For example, a photocoagulative laser such as a diode laser (400-800 nanometer range) may be selected to cause contraction of the lamina cribrosa thereby thinning it and putting it under tension. This tension facilitates incision of the lamina cribrosa by selecting and utilizing a photodisruptive laser (such as a nanosecond, 1064 nm Nd:YAG laser) or by selecting and using a photoablative laser (such as a femtosecond Nd:YAG). A photocoagulative laser may also be selected and used following decompression of the CRVO to stop any bleeding caused by the treatment.

If the patient has a large optic cup, incision of the lamina cribrosa may be all that is necessary to decompress the compartment compressing the central retinal vein. If the patient has a small optic cup, incision of a portion of the substance of the patient's optic nerve may be necessary in addition to incision of the lamina cribrosa. Although it may be possible to incise said tissues with a single high-power application of laser energy, multiple passes using lower energies are preferred. By selecting the least possible amount of laser energy to accomplish decompression, collateral damage to surrounding structures such as the central retinal artery and vein are minimized. When incision of the optic nerve head is necessary, multiple low-energy laser application will help minimize visual field loss from optic nerve damage. When practical, the nerve head is incised at the nasal midline in order to minimize visual field loss and avoid damage to macular nerve fibers.

The method ends with step 412.

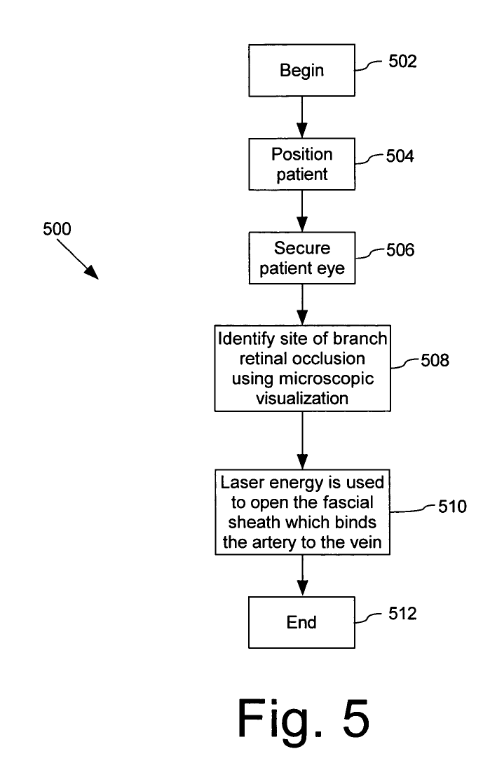

Referring now to FIG. 5, shown is a flow diagram of a method of treatment 500 for an ocular compartment syndrome such as a branch retinal vein occlusion that is useful for understanding the invention. The method begins with step 502 and continues with step 504.

In step 504, the patient is positioned beneath an operative microscope 110 and one or more of lasers 108, 109, and 111. In step 506, the operative eye 102 is stabilized by selecting and positioning a fixation ring 104 or other fixation device on the operative eye 102. In step 508, the site of the branch retinal vein occlusion (generally an arteriovenous crossing) is identified using microscopic visualization. In step 510, laser energy is used to open the fascial sheath which binds the artery to the vein at the site of the branch retinal vein occlusion identified in step 508. In this step, a single photocoagulative, photodisruptive, or photoablative laser may be selected and used for this purpose. However, in the preferred embodiment of the invention, a combination of one or more laser types is selected in order to achieve the desired effect.

For example, a photocoagulative laser such as a diode laser (400-800 nanometer range) may be selected and used to cause contraction of the internal limiting membrane (ILM) that makes up the arterio-venous fascial sheath, thereby thinning it and placing it under tension. This tension facilitates incision of the sheath with a photodisruptive laser. A photodisruptive laser that can be selected includes a nanosecond, 1064 nm Nd:YAG laser. In other embodiments of the invention, a photoablative laser could be selected such as a femtosecond, 1064 nm Nd:YAG laser. This type of Nd:YAG laser ablates the fascial sheath and/or Internal Limiting Membrane (ILM) surrounding the area of retinal venous constriction, thus, restoring venous blood flow without disrupting the full thickness of the underlying retinal vessels and surrounding structures. A photocoagulative laser can also be selected and used following decompression of the BRVO to stop any bleeding caused by the treatment. Although it may be possible to decompress the branch retinal vein with a single high-power application of laser energy, multiple passes using lower energies are preferred. By selecting and using the least possible amount of laser energy to accomplish decompression, collateral damage to the affected vein, the adjacent artery, and the surrounding artery are minimized.

The method ends with step 512.

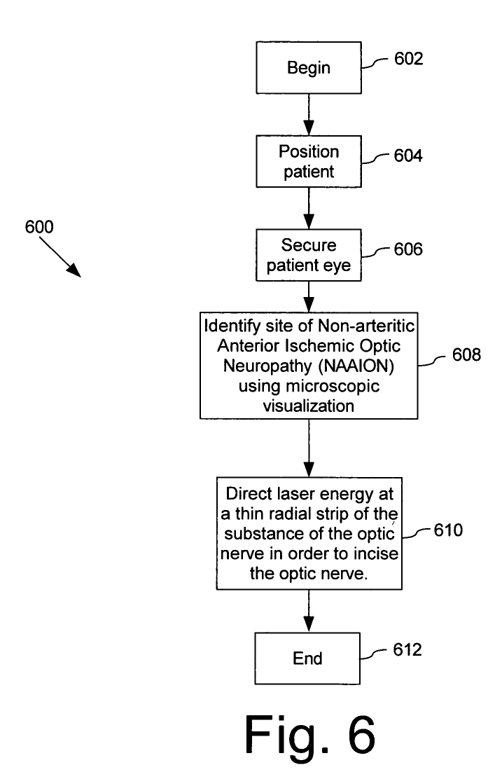

Referring now to FIG. 6, shown is a flow diagram of a method of treatment 600 for an ocular compartment syndrome such as a Non-Arteritic Anterior Ischemic Optic Neuropathy (NAAION) that is useful for understanding the invention. The method begins with step 602 and continues with step 604. In step 604, the patient is positioned beneath an operative microscope 110 and one or more of lasers 108, 109, and 111. In step 606, the operative eye 102 is stabilized by selecting and positioning a fixation ring 104 or other fixation device on the operative eye 102. In step 608, microscopic visualization is used to identify the patient's optic nerve and, if possible, any areas of obvious ischemia related to the NAAION.

In step 610, laser energy is directed at a thin radial strip of the substance of the optic nerve in order to incise the nerve in much the same way a steel blade is used to perform a traditional radial optic neurotomy. Whenever practical, the nerve head is incised at the nasal midline in order to minimize visual field loss and avoid damage to macular nerve fibers. Alternatively, the neurotomy can be performed in an area that already shows evidence of ischemia, so as to minimize visual field loss. A single photocoagulative, photodisruptive, or photoablative laser may be used for this purpose. In the preferred embodiment of the invention, a combination of one or more laser types is selected in order to achieve the desired effect.

For example, a photocoagulative laser such as a diode laser (400-800 nanometer range) may be selected and used to cause contraction of the target tissue thereby thinning it and putting it under tension. This tension facilitates incision of the tissue with a photodisruptive laser (such as a nanosecond, 1064 nanometer Nd:YAG laser) that can be selected and/or a photoablative laser (such as a femtosecond, 1064 nm Nd:YAG) that can also be selected. A photocoagulative laser may also be selected and used following decompression of the NAAION to stop any bleeding caused by the treatment. Although it may be possible to incise said tissues with a single high-power application of laser energy, multiple passes using lower energies are preferred. By selecting the least possible amount of laser energy to accomplish decompression, visual field loss due to optic nerve damage is minimized.

The method ends with step 612.

Papilledema is another ocular compartment syndrome which is amenable to treatment with the proposed method and apparatus. Unlike the previously described ocular compartment syndromes, in papilledema, the source of compressive force comes from elevated cerebrospinal fluid (CSF) pressure. This force compresses the optic nerve and results in impaired blood flow to the nerve as well as axoplasmic stasis. Lowering of intracranial pressure can be achieved through traditional means such as a ventriculoperitoneal shunt. Because ventricular shunting requires brain surgery, however, a less invasive treatment would be desirable and highly preferable. Incision of the optic nerve sheath through a medial or lateral orbitotomy can also be used to decompress this compartment syndrome although this also requires significant surgical trauma.

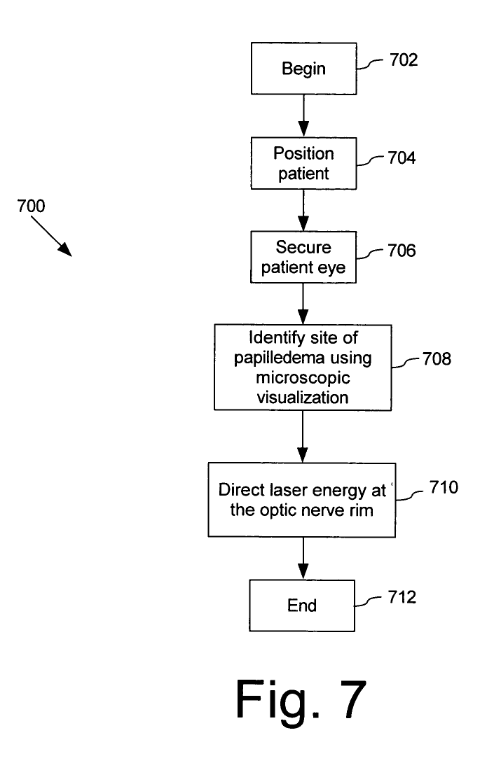

Referring now to FIG. 7, shown is a flow diagram of a method of treatment 700 for an ocular compartment syndrome such as a papilledema that is useful for understanding the invention. The method begins with step 702 and continues with step 704. In step 704, the patient is positioned beneath an operative microscope 110 and one or more of lasers 108, 109, and 111. In step 706, the operative eye 102 is stabilized by selecting and positioning a fixation ring 104 or other fixation device on the operative eye 102. In step 708, microscopic visualization is used to identify the patient's optic nerve and/or optic nerve sheath. In step 710, laser energy is directed at the optic nerve rim in order to penetrate into the space where CSF is present under pressure. CSF is vented into the vitreous cavity where it can be reabsorbed. A single photocoagulative, photodisruptive, or photoablative laser may be selected and used for this purpose. However, in the preferred embodiment of the invention, a combination of one or more laser types is preferred to be selected in order to achieve the desired effect.

For example, a photocoagulative laser such as a diode laser (400-800 nanometer range) may be selected and used to cause contraction of the optic nerve rim thereby thinning it and putting it under tension. This tension facilitates incision of the nerve rim with a photodisruptive laser such as a nanosecond, 1064 nm Nd:YAG laser. In other embodiments of the invention, a photoablative laser may be selected, such as a femtosecond 1064 nm Nd:YAG laser to create photoablative effects. The photocoagulative laser may also be used to control any bleeding caused by the treatment. Although it may be possible to incise said tissue with a single high-power application of laser energy, multiple passes using lower energies are preferred. By using the least possible amount of laser energy to accomplish decompression, damage to the optic nerve is minimized. When practical, the nerve head is incised at the nasal midline in order to minimize visual field loss and avoid damage to macular nerve fibers.

The method ends with step 712.

All of the apparatus, methods and compositions disclosed and claimed herein can be made and executed without undue experimentation in light of the present disclosure. While the invention has been described in terms of preferred embodiments, it will be apparent to those of skill in the art that variations may be applied to the apparatus, methods and sequence of steps of the method without departing from the concept, spirit and scope of the invention. More specifically, it will be apparent that certain components may be added to, combined with, or substituted for the components described herein while the same or similar results would be achieved. All such similar substitutes and modifications apparent to those skilled in the art are deemed to be within the spirit, scope and concept of the invention as defined by the appended claims. Accordingly, the exclusive rights sought to be patented are as described in the claims below.

We claim:

1. A method for the treatment of ocular compartment syndromes comprising:
directing laser energy at an ocular tissue responsible for an ocular compartment syndrome.

2. The method according to claim 1, wherein said directing step further comprises selecting said laser energy to produce an effect on said ocular tissue selected from the group consisting of (a) a photoablative effect, (b) a photodisruptive effect, and (c) a photocoagulative effect.

3. The method according to claim 2, wherein said directing step further comprises directing a first type of laser energy at said ocular tissue for achieving a first tissue effect, and subsequently directing a second type of laser energy at said ocular tissue for achieving a second tissue effect, and further comprising selecting said first type of laser energy to be different in at least one characteristic as compared to said second type of laser energy.

4. The method according to claim 3, further comprising directing a third type of laser energy at said ocular tissue, and selecting said third type of laser energy to be different in at least a second characteristic as compared to said first and said second type of laser energy.

5. The method according to claim 4, further comprising selecting said first and said second characteristic from the group consisting of a wavelength, a pulse duration, and a power level.

6. The method according to claim 1, further comprising selecting said ocular tissue to include a site of a central retinal vein occlusion.

7. The method according to claim 6, further comprising selecting said ocular tissue to include a portion of an optic nerve.

8. The method according to claim 1, further comprising selecting said ocular tissue to be a site of a branch retinal vein occlusion.

9. The method according to claim 8, further comprising selecting said ocular tissue from the group consisting of a fascial sheath and an internal limiting membrane (ILM) surrounding an area of retinal venous constriction.

10. The method according to claim 1, further comprising selecting said ocular tissue to be a site of a non-arteritic anterior ischemic optic neuropathy (NAAION).

11. The method according to claim 10, further comprising selecting said ocular tissue from the group consisting of an optic nerve and an optic nerve sheath.

12. The method according to claim 1, further comprising selecting said ocular tissue to be a site of papilledema.

13. The method according to claim 12, further comprising selecting said ocular tissue from the group consisting of an optic nerve and an optic nerve sheath.

14. The method according to claim 13, wherein said directing step further comprises applying said laser energy to said optic nerve rim.

15. The method according to claim 1, further comprising using Optical Coherence Tomography (OCT) to augment microscopic visualization of a treatment area.

16. A method for the treatment of ocular compartment syndromes comprising:
positioning a patient so that ocular tissue can be observed with a microscope;
identifying the site of an ocular compartment syndrome; and
directing laser energy at an ocular tissue to relieve at least one symptom of an ocular compartment syndrome.

17. A method for the treatment of ocular compartment syndromes comprising:
positioning a patient so that ocular tissue can be exposed to laser energy; and
directing said laser energy at an ocular tissue causing an ocular compartment syndrome.
